(19) United States
(12) Patent Application Publication
(10) Pub. No.: US 2021/0069469 A1
(43) Pub. Date: Mar. 11, 2021

(54) CATHETER COMPRISING FLEXIBLE PRINTED CIRCUIT BOARD AND MANUFACTURING METHOD FOR FLEXIBLE CIRCUIT BOARD

(71) Applicant: Carl Freudenberg KG, Weinheim (DE)

(72) Inventor: Benno Schmied, Ludwigshafen (DE)

(21) Appl. No.: 17/015,099

(22) Filed: Sep. 9, 2020

(30) Foreign Application Priority Data
Sep. 10, 2019 (EP) 19196443.6

(51) Int. Cl. A61M 25/00; H05K 1/02; H05K 3/00; A61B 5/042; A61B 5/00; A61N 1/362; A61N 1/05

(52) U.S. Cl. CPC A61M 25/0082; H05K 1/028; H05K 3/0044; A61B 5/042; A61B 5/6852; A61B 2562/166; A61N 1/05; H05K 2203/0228; A61B 2562/0209; A61B 2562/164; A61N 1/362

(57) ABSTRACT

A catheter includes: an elongate, hollow, and flexible tube; at least one electrode for carrying out a diagnosis or therapy in a human body, the at least one electrode being arranged at an end region of the tube; and an electrical terminal attached at an other end region of the tube for electrically connecting the catheter to a control device. The at least one electrode and the electrical terminal are connected to one another in an electrically conducting manner by way of conductive tracks on a flexible printed circuit board. The flexible printed circuit board is arranged in the tube. The printed circuit board has a shape of a long ribbon, and the ribbon is pleated.

### CROSS-REFERENCE TO PRIOR APPLICATION

Priority is claimed to European Patent Application No. EP 19196443.6, filed on Sep. 10, 2019, the entire disclosure of which is hereby incorporated by reference herein.

### FIELD

The invention relates to a catheter comprising a flexible printed circuit board, and to a method for manufacturing a flexible printed circuit board.

### BACKGROUND

A wide variety of catheter designs are known from the prior art, which are constructed in accordance with the medical application thereof. This also includes electrode-carrying catheters, such as are described, for example, in DE 694 01 562 T2. So-called “mapping catheters” are also known, for example, for the 3-dimensional detection of the anatomy of a ventricle, which must include a high number of electrodes.

In the catheters known from the prior art, high production costs arise because the conductive tracks leading to the electrodes have to be accommodated in the catheter tube in a complex manner. It is further problematic that, due to the materials used, a limitation can result with respect to the bending radius of the catheter and, consequently, a restriction in the use thereof.

### SUMMARY

In an embodiment, the present invention provides a catheter, comprising: an elongate, hollow, and flexible tube; at least one electrode configured to carry out a diagnosis or therapy in a human body, the at least one electrode being arranged at an end region of the tube; and an electrical terminal attached at an other end region of the tube for electrically connecting the catheter to a control device, wherein the at least one electrode and the electrical terminal are connected to one another in an electrically conducting manner by way of conductive tracks on a flexible printed circuit board, wherein the flexible printed circuit board is arranged in the tube, and wherein the printed circuit board has a shape of a long ribbon, and the ribbon is pleated.

### BRIEF DESCRIPTION OF THE DRAWINGS

The present invention will be described in even greater detail below based on the exemplary figures. The invention is not limited to the exemplary embodiments. Other features and advantages of various embodiments of the present invention will become apparent by reading the following detailed description with reference to the attached drawings which illustrate the following:

FIG. 1 shows a catheter according to the invention;

FIG. 2a shows a material-saving and cost-effective manufacture of a printed circuit board;

FIG. 2b shows an alternative material-saving and cost-effective manufacture of a printed circuit board;

FIG. 3 shows a printed circuit board in crimped form;

FIG. 4 shows a crimped printed circuit board inside a tube; and

FIG. 5 shows the process of pleating a printed circuit board.

### DETAILED DESCRIPTION

In an embodiment, the present invention provides a cost-effective and, at the same time, flexible catheter and to at least partially eliminate the disadvantages of the prior art. In an embodiment, the present invention provides a cost-effective manufacturing method for flexible printed circuit boards that can be used in such a catheter.

The use of a flexible printed circuit board comprising conductive tracks was found to be advantageous according to the invention:

The catheter has the shape of an elongate, hollow and flexible tube, which could also be referred to as a hose. It comprises a hollow catheter. The catheter has at least one electrode for carrying out a diagnosis, for example a measurement such as an electrophysiological examination, or for carrying out a therapy, for example an operation such as a catheter ablation, in the human body in each case. The at least one electrode is arranged at an end region of the tube, and an electrical terminal is attached at the other end region of the tube for electrically connecting the catheter to a control device, such that a current-conducting and data-transmitting connection exists between the at least one electrode and the electrical terminal.

According to the invention, the printed circuit board has the shape of a long ribbon. This has the advantage that a single printed circuit board can then suffice to establish the connection between the electrode and the electrical terminal across the entire length of the tube.

According to the invention, the ribbon is pleated, i.e., is provided with a zigzag fold.

Such a deformed printed circuit board can follow bending movements of the tube particularly well and tolerates even tight bending radii, and high strain acting on the printed circuit board during the bending movement and breaking the conductive tracks can be prevented.

The at least one electrode and the electrical terminal are advantageously connected to one another in an electrically conducting manner by means of conductive tracks on at least one flexible printed circuit board. The flexible printed circuit board is in particular film-like and pliable, but not necessarily extensible. Polyimide (PI) can be used as the material, for example. The conductive tracks can in particular be made of copper or copper alloys. According to the invention, the flexible printed circuit board is arranged in the tube, i.e., inside the hollow tube.

Such a catheter is particularly cost-effective to manufacture, since no individual current conductors have to be run. The use of a flexible printed circuit board in the interior of the tube also renders the catheter very flexible.

It appears to be particularly advantageous if the ribbon is severed out of a flexible printed circuit board in a helical manner as a ribbon having a constant width. Such a flexible printed circuit board is particularly cost-effective to produce. More details will be apparent below from the description of the manufacturing method according to the invention.

It has proven to be particularly advantageous if the ribbon is crimped, i.e., wound in a spiral-shaped manner in the longitudinal axial direction thereof.

If the printed circuit board is additionally twisted about the longitudinal axis of the catheter, the degrees of freedom of the printed circuit board are further increased, and damage to the printed circuit board is prevented even better.

The invention also relates to a method for manufacturing a flexible printed circuit board in the form of a long ribbon, in particular for a catheter as described above, comprising the following steps:

a. providing a flexible carrier plate;

b. applying conductive tracks to the flexible carrier plate so as to produce a planar printed circuit board;

c. severing a ribbon out of the planar printed circuit board, wherein the cutting line has the shape of a helical spiral, i.e., an Archimedean spiral. In an alternative method variant, the cutting line can have a U-shape, including two legs and a bend connecting the legs.

As an additional optional step, the ribbon can be extended by applying opposing tensile forces at the ends of the ribbon. The spiral is thereby pulled apart to form a long, crimped ribbon.

d. pleating, i.e., folding the ribbon in a zigzag-shaped manner.

Such a manufacturing method for producing a flexible printed circuit board is particularly cost-effective since, on the one hand, it saves material and, on the other hand, is fast, because a device for applying the conductive tracks and a tool for severing only have to reach a comparatively small area, i.e., only small relative movements have to be implemented.

It is further advantageous that crimping of the ribbon-shaped printed circuit board results automatically, and thus good flexibility of the printed circuit board is achieved.

It has proven to be particularly advantageous if the spiral has a constant pitch, so that a ribbon having a constant width is created. The spiral is an involute of a circle, for example.

In an advantageous refinement of the method according to the invention, the severing is effectuated by stamping or cutting, in particular flame cutting or jet or beam cutting, such as, for example, water jet cutting or laser beam cutting.

The invention also relates to the use of a catheter as described above as a cardiac catheter, which is used in the diagnosis or therapy of the human heart.

The invention will now be explained in more detail using the accompanying figures. Corresponding elements and components are provided with the same reference symbols in the figures. For the sake of better clarity of the figures, a presentation that is true to scale has been dispensed with.

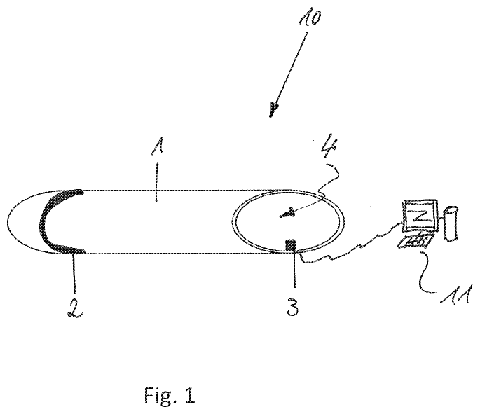

FIG. 1 shows a catheter 10 according to the invention, which has the shape of an elongate flexible tube 1. At least one electrode 2 is arranged at the one end region thereof An electrical terminal 3 is attached to the other end region thereof. The at least one electrode 2 is connected by way of conductive tracks 5 on an elastically extensible printed circuit board 4 within the tube 1 to the electrical terminal 3 in a data-transmitting and current-conducting manner. A control device 11, which is used to control the catheter 10, is connected to the terminal 3.

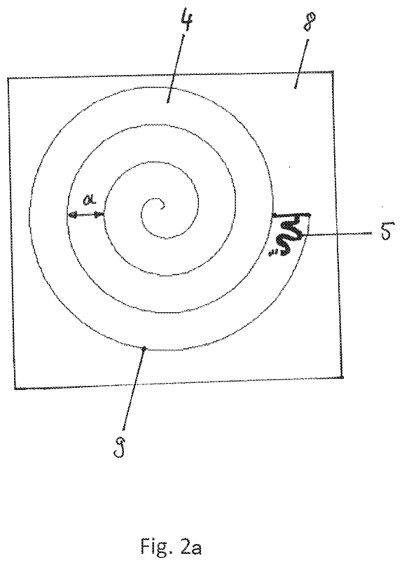

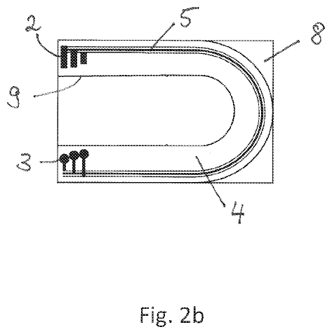

FIG. 2a shows a material-saving and cost-effective option for manufacturing a printed circuit board 4. First, a flexible carrier plate 8 is provided. Subsequently, conductive tracks 5 are applied to the flexible carrier plate 8 so as to produce a planar printed circuit board 4. The conductive tracks 5 are only hinted at in the figure. A ribbon is then severed from the planar printed circuit board 4, wherein the cutting line 9 has the shape of a helical spiral, i.e., an Archimedean spiral. The pitch a of the spiral is constant such that a ribbon having a constant width is created.

FIG. 2b shows an alternative material-saving and cost-effective option for manufacturing a printed circuit board 4 that is particularly advantageous if a plurality of electrodes are provided at a catheter. First, a flexible carrier plate 8 is provided. Subsequently, the conductive tracks 5 are applied to the flexible carrier plate 8 so as to produce a planar printed circuit board 4. The conductive tracks 5 are only indicated as lines in the figure, even though in practice they preferably have a meander shape. The electrodes 2 and electrical terminals 3 can also be applied. A ribbon is then severed out of the planar printed circuit board 4, wherein the cutting line 9 has the shape of an arrangement of a straight line, a semicircle and another straight line, that is to say, a U-shape comprising two legs and a bend connecting the legs. Electrodes 2 and electrical terminals 3 are located in the straight regions and can thus be produced more easily.

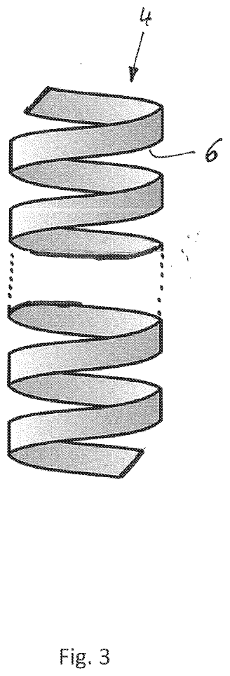

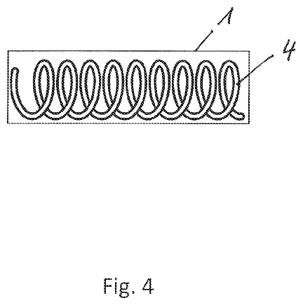

FIG. 3 shows a printed circuit board 4 in the shape of a long ribbon with a crimp 6, which results from the manufacturing method shown in FIG. 2. Such a crimped printed circuit board 4 can be positioned inside a tube 1 to create a catheter 10, as shown in FIG. 4.

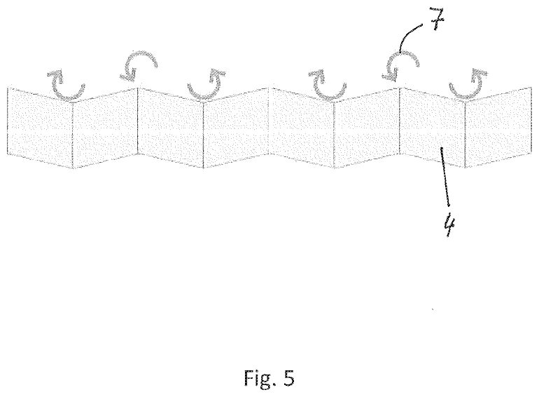

FIG. 5 shows the process of pleating a ribbon-shaped printed circuit board 4. The pleating, i.e., creation of a pleat 7, is carried out by folding the printed circuit board 4 in zigzag manner.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

### LIST OF REFERENCE SIGNS

1 Tube
2 Electrode
3 Terminal
4 Printed circuit board
5 Position of conductive track
6 Crimp
7 Pleat
8 Carrier plate
9 Cutting line
10 Catheter
11 Control device
a Pitch

What is claimed is:

1. A catheter, comprising:
an elongate, hollow, and flexible tube;
at least one electrode configured to carry out a diagnosis or therapy in a human body, the at least one electrode being arranged at an end region of the tube; and
an electrical terminal attached at an other end region of the tube for electrically connecting the catheter to a control device,
wherein the at least one electrode and the electrical terminal are connected to one another in an electrically conducting manner by way of conductive tracks on a flexible printed circuit board,
wherein the flexible printed circuit board is arranged in the tube, and
wherein the printed circuit board has a shape of a long ribbon, and the ribbon is pleated.

2. The catheter according to claim 1, wherein the ribbon is severed out of a flexible printed circuit board in a helical manner as a ribbon having a constant width.

3. The catheter according to claim 1, wherein the ribbon is crimped.

4. A method for manufacturing a flexible printed circuit board in a shape of a long ribbon for the catheter according to claim 1, the method comprising the following steps:
a. providing a flexible carrier plate;
b. applying conductive tracks to the flexible carrier plate so as to produce a planar printed circuit board;
c. severing, along a cutting line, a ribbon out of the planar printed circuit board, the cutting line of having a shape of a helical spiral or a U-shape; and
d. pleating the ribbon.

5. The method according to claim 4, wherein the helical spiral has a constant pitch.

6. The method according to claim 4, wherein the severing is effectuated by stamping or cutting.
